(19) United States
(12) Patent Application Publication
(10) Pub. No.: US 2012/0122935 A1
(43) Pub. Date: May 17, 2012

(54) COMPOSITIONS AND METHODS FOR TREATING NECROTIZING ENTEROCOLITIS

(75) Inventors: Peter J. Giannone, Dublin, OH (US); John A. Bauer, Westerville, OH (US)

(73) Assignee: NATIONWIDE CHILDREN'S HOSPITAL, INC., Columbus, OH (US)

(21) Appl. No.: 13/211,229

(22) Filed: Aug. 16, 2011

Related U.S. Application Data
(63) Continuation of application No. 12/253,888, filed on Oct. 17, 2008, now abandoned.
(60) Provisional application No. 60/980,750, filed on Oct. 17, 2007.

(51) Int. Cl. A61K 31/455; A01P 1/00; C07D 213/82

(52) U.S. Cl. 514/355; 546/316

(57) ABSTRACT

The present invention relates to a method for treating or preventing necrotizing enterocolitis (NEC) in a human neonate in need thereof, comprising administering to the neonate a pharmaceutically effective amount of a composition comprising a poly(ADP-ribose) synthetase/polymerase (PARP) inhibitor. Also contemplated herein is an infant food or treatment composition comprising a PARP inhibitor in an amount that is 5 to 500 times greater than a daily recommended intake dosage for the PARP inhibitor.

### RELATED APPLICATIONS

This application is a continuation of U.S. application Ser. No. 12/253,888, filed on Oct. 17, 2008, which claims the benefit of U.S. Provisional Application No. 60/980,750, filed on Oct. 17, 2007, the entire contents of which are incorporated herein by reference.

### BACKGROUND

Necrotizing enterocolitis (NEC) is the most common gastrointestinal disease of infants, afflicting 5-10% of all infants born at ≦1500 grams birth weight or less than 30 weeks gestational age. Although most babies who develop NEC are born prematurely, approximately 10% of babies with NEC are full-term infants. The incidence of NEC has actually increased in the era of surfactant replacement therapy probably because of increased aggressive care of infants born weighing ≦800 grams and fewer premature infants succumb to respiratory distress syndrome. NEC has a 30-40% mortality rate and is associated with high morbidity in survivors.

Necrotizing enterocolitis is a disease that is unique to the infant population and is well recognized by physicians and researchers as different and separate from various other forms of intestinal disease. This is because the setting and course of disease progression is unique, the patient population is very different from other age groups (e.g. the intestine and other vital organs are very immature and still in development in preterm neonates, both in structure and function). For these reasons and at this time there is no known therapeutic strategy that has been shown to treat or prevent necrotizing colitis.

The current treatment for infants that develop NEC is solely supportive as enteral feedings are withheld and intravenous fluids are given. Respiratory and cardiovascular systems are supported as clinically indicated and antibiotics are administered. Indications for surgical intervention include bowel perforation and/or indications of bowel necrosis. Although surgical treatment involves resection of dead bowel, peritoneal drainage for the smallest infants has recently been used in lieu of emergent bowel resection. Therefore, it is desirable to develop therapeutic agents for the prevention or treatment of NEC.

### SUMMARY

The present invention relates to a method of treating or preventing necrotizing enterocolitis (NEC) in a human neonate in need thereof. The method includes administering to the neonate a pharmaceutically effective amount of a composition comprising a poly(ADP-ribose) synthetase/polymerase (PARP) inhibitor.

In one embodiment, the neonate has NEC and the PARP inhibitor is administered in an amount sufficient to prevent or slow the progression of NEC from an earlier stage to a later stage of the disease.

In another embodiment, the neonate is at risk of developing NEC and the PARP inhibitor is administered in an amount sufficient to prevent the clinical onset of NEC in a neonate.

In any of the above embodiments, the neonate may be preterm.

In some embodiments, the PARP inhibitor is selected from the group consisting of: nicotinamide, 3-aminobenzamide, picolinamide, 5-methyl nicotinamide, methylxanthines, thymidine, benzamide, 3-methoxybenzamide, 4-aminobenzamide, 2-aminobenzamide, pyrazinamide, theobromine, theophylline, 3-aminophtalhydrazide, and 1,5-dihydroxyisoquinoline, or a mixture thereof.

In some embodiments, the composition is administered via an oral, enteral, rectal, intravenous, intra-arterial, intramuscular, intraperitoneal or subcutaneous route.

In some embodiments, the composition further comprises a pharmaceutically acceptable carrier or excipient.

Also contemplated herein is a method for treating a human neonate having necrotizing enterocolitis (NEC) or being at risk of developing NEC. The method includes administering to the neonate a pharmaceutically effective amount of a composition comprising nicotinamide.

In some embodiments, the neonate has NEC and the nicotinamide is administered in an amount sufficient to prevent or slow the progression of NEC from an earlier stage to a later stage of the disease. In some examples, the neonate has Grade 1 or Grade 2 NEC.

In other embodiments, the neonate is at risk of developing NEC and the PARP inhibitor is administered in an amount sufficient to prevent the clinical onset of NEC in a neonate.

In any of the above mentioned embodiments, the neonate can be preterm.

In some embodiments, the therapeutically effective amount of nicotinamide is from 5-500 mg/Kg.

The compositions contemplated herein may be administered via an oral, enteral, rectal, intravenous, intra-arterial, intramuscular, intraperitoneal or subcutaneous route.

In some embodiments, the compositions further comprises a pharmaceutically acceptable carrier or excipient.

In another aspect, the invention relates to an infant food or treatment composition comprising a PARP inhibitor in an amount that is 5 to 500 times greater than a daily recommended intake dosage for the PARP inhibitor.

In some embodiments, the composition is a synthetic infant formula for preterm neonates. In some examples, the synthetic infant formula for preterm neonates includes, as the PARP inhibitor, nicotinamide in an amount that is 17-4500 mg/L of said formula.

In other embodiments, the composition is a dietary food supplement for neonates at risk of developing NEC. In some examples, dietary food supplement contains, as the PARP inhibitor, nicotinamide in an amount that is 17-4500 mg/L.

In other embodiments, the composition is a pharmaceutical preparation for administration via a non-oral route. In some examples, the PARP inhibitor in the composition is nicotinamide.

### BRIEF DESCRIPTION OF FIGURES

FIG. 1 is a proposed scheme involving PARP activation in necrotizing enterocolitis.

FIG. 2 is a schematic representation of the actions of PARP, which causes polymerization of ADP-ribose, and is inhibited by nicotinamide and other known inhibitors

FIG. 3 shows representative photomicrographs of progressive intestinal tissue destruction in necrotizing enterocolitis, and grades of disease progression. At Grade 0 minimal evidence of villous damage in healthy tissue; in Grades 1 and 2 progressive damage and degradation of the tips of villi (the inner region of the mucosal layer) leading to reductions in internal barrier function and tissue integrity; in Grade 3 complete or near complete destruction of the mucosal layer; Grade 4 loss of mucosal layer, damage and destruction of the underlying muscle (surosal) layer.

FIG. 4 shows representative photomicrographs of both human intestinal tissues and rat intestinal tissues. The upper panels show healthy tissue in both species and the lower panel shows NEC specimens in both species. These results demonstrate that the preclinical neonatal rat model of necrotizing enterocolitis mimics the human disease state. Intestinal tissues from the animal model are identical to human tissue pathology at the stages of NEC progression.

FIG. 5 is a chart showing increased reactive nitrogen species in intestinal mucosal layer during necrotizing enterocolitis. Nitrated tissue proteins (the amino acid 3-nitrotyrosine (NT)) were measured in the proximal villus region using immunohistochemistry, and were shown to be increased in NEC tissues. (IOD; integrated optical density, a measure of the concentration of the measured analyte.)

FIG. 6 shows evidence of PARP activation in NEC tissues. PARP was increased in the earliest NEC grades (top panel—showing the prevalence of PARP—1 in rat illeal tissues with Grade 1 and Grade 2 NEC using immunohistochemical staining) The amounts of PARP in intestinal mucosal regions during NEC were highly correlated to the amount of reactive nitrogen species (bottom panel showing 3-NT levels in the same tissue regions). These data are consistent with tissue oxidation and PARP activation being linked in the mucosal layer during NEC progression.

FIG. 7 is a demonstration of PARP activation in human NEC tissue. Immunohistochemistry was used to observe the prevalence of PARP in human intestinal tissues resected from necrotizing colitis patients. PARP was readily detectable in the mucosal region. (H&E=hematoxylin and eosin stain, α-PARP=anti-PARP antibody)

FIG. 8 depicts rat NEC model survival proportions and demonstrates that the treatment of neonatal rat pups in the preclinical model with nicotinamide improves survival.

FIG. 9—(A) shows photomicrographs of epithelial restitution in FHS741NT cells following wounding. Images taken at 0, 24, and 48 hrs after wounding. (B) Percent wound closure at 0, 24, and 48 hrs. (* P<0.01 vs 0 hr wound by One-Way ANOVA, Dunnett's post-test.)

FIG. 10 shows percent of epithelial restitution 48 hrs after wounding with or without lipopolysaccharide (LPS) treatment. (* P<0.01 vs. untreated; Student's t-test).

FIG. 11 shows PAR immunostaining in intestinal epithelial cells (IECs) after wounding. A and C) fluorescence images of anti-PAR staining in untreated (A) and LPS treated (C) cells. B and D) DIC image of same fields of view shown in A and C overlaid with the fluorescence staining Note the restriction of the staining pattern to the nucleus and the paucity of anti-PAR staining in the untreated cells. E and F) are quantifications of the fluorescence of PAR for wounded IECs treated with 100 ug/ml LPS and 200 ug/ml LPS respectively. PAR is significantly elevated in the LPS treated IECs. (*=p<0.005; Student t-test).

FIG. 12 shows nuclear α-PARP immunofluoresence at 10 min post wounding. PARP immunostaining is significantly elevated after wounding of fetal IECs and the addition of LPS. This model is in fetal IECs that are unique from adult IECs.

FIG. 13 shows percentage of animals with intestinal NEC Grade of 2 or greater following administration of either vehicle or enteral LPS. (* P=0.0002; Student's t-test.)

FIG. 14 shows the effect of LPS, 50 mg/ml of nicotinamide plus LPS, and untreated fetal intestinal epithelial cells after wounding in the in vitro model of immature intestinal wound restitution. Nicotinamide improves wound healing in a fetal IEC model of intestinal injury.

### DETAILED DESCRIPTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The present invention relates to the treatment and prevention of neonatal necrotizing enterocolitis through the inhibition of the action of poly(ADP-ribose) synthetase/polymerase (PARP). Also contemplated are compositions containing high-dose PARP inhibitors, such as infant formulas, infant food supplements, or pharmaceutical compositions for the treatment or prevention of NEC.

The present invention is based on our findings that PARP inhibitors are protective against intestinal injury in a newborn rat model of NEC. Our studies are the first to establish a link between PARP activation and neonatal NEC.

### DEFINITIONS

“Infant,” as used herein, refers to any human baby within the first 2 years of life.

“Neonatal NEC” or “NEC” are used interchangeably and mean the disorder necrotizing enterocolitis, as described below, which can afflict a neonate.

“Neonate,” as used herein, refers to any human baby from birth through the first four months of extrauterine life.

“Neonate at risk of developing NEC” refers to neonates who are premature (less than 37 weeks gestation) and those who are full-term but are in an at risk environment or exhibit a sign of having been exposed to stress, examples include, but are not limited to, prenatal asphyxia, shock, sepsis, congenital heart disease, hypoxemia, hypertension, catheterization of umbilical vessels, intrauterine growth retardation, babies who are fed concentrated formulas or who have received blood exchange transfusions.

A “PARP inhibitor” refers to a compound that is capable of reducing the activity of PARP by at least 20%.

A “pharmaceutically acceptable carrier or excipient” as used herein refers to any carrier, diluent, excipient, suspending agent, lubricating agent, adjuvant, vehicle, delivery system, emulsifier, disintegrant, absorbent, preservative, surfactant, colorant, flavorant, or sweetener.

Such a carrier or excipient is useful in preparing a pharmaceutical composition that is generally safe, non-toxic and neither biologically nor otherwise undesirable, and includes a carrier or an excipient that is acceptable for human pharmaceutical use. “A pharmaceutically acceptable carrier/excipient” as used in the specification and claims includes both one and more than one such excipient.

The terms “premature” and “preterm” are used interchangeably and refer to a neonate born before 37 weeks gestation. At birth, a baby is classified as one of the following: premature or preterm (less than 37 weeks gestation); term or full-term (37 to 42 weeks gestation); post-term (born after 42 weeks gestation).

“Preventing” or “preventing the clinical onset of NEC” refers to either (i) causing the clinical manifestations of NEC not to develop in a neonate that may be at risk of developing NEC but does not yet experience or display the clinical signs and symptoms of NEC; or (ii) delaying the onset of NEC or reducing the severity of NEC once it develops.

“Preventing or slowing the progression of NEC from an earlier stage to a later stage of the disease” refers to preventing or slowing the progression of NEC from Grade 1 to Grade 2, from Grade 2 to Grade 3, or from Grade 3 to Grade 4.

A “therapeutically effective amount” means the amount of a compound that, when administered to a neonate for treating or preventing NEC, is sufficient to effect such treatment or prevention as defined herein. The “therapeutically effective amount” will vary depending on the compound, the disease severity and the age, weight, etc., of the neonate to be treated. In some embodiments, such a therapeutically effective amount is the amount of PARP inhibitor needed to inhibit PARP activity by at least 20% in tissue affected by NEC as compared to an untreated control.

“Treating NEC” refers to (i) inhibiting NEC, i.e., slowing, arresting or reducing the development of NEC or its clinical manifestations, including slowing, reducing or preventing the progression of NEC from an earlier stage to a later stage of the disease; (ii) relieving NEC, i.e., causing regression of NEC or its clinical symptoms; or (iii) increasing the rate of healing in the intestine of a neonate affected by NEC.

Necrotizing Enterocolitis (NEC)

Necrotizing enterocolitis is the death of intestinal tissue. It primarily affects premature infants or sick newborns. Its causes are not known. Necrotizing enterocolitis occurs when the lining of the intestinal wall dies and the tissue falls off.

Babies considered to be at risk for NEC are those who are premature (less than 37 weeks gestation) and those who are full-term but are in an at risk environment or exhibit a sign of having been exposed to stress, e.g., prenatal asphyxia, shock, sepsis, congenital heart disease, hypoxemia, hypertension, catheterization of umbilical vessels, intrauterine growth retardation, babies who are fed concentrated formulas or who have received blood exchange transfusions, etc. Overall, however, preterm birth is the most identifiable risk factor, especially for extremely low birth weight infants (ELBW) (born weighing ≦1500 grams) or gestational age ≦30 weeks. Thus, NEC is likely to be related to an immature gastrointestinal tract that is not prepared for postnatal conditions.

The presence and severity of NEC is graded using the staging system of Bell et al., J. Ped. Surg., 15:569 (1980) as shown in Table I.

TABLE 1
Clinical manifestations of different NEC stages

| | |
|---|---|
| Stage I | Any one or more historical factors producing perinatal stress |
| (Suspected | Systemic manifestations - temperature instability, lethargy, |
| NEC) | apnea, bradycardia |
| | Gastrointestinal manifestations - poor |
| | feeding, increased pregavage residuals, emesis (may be |
| | bilious or test positive for occult blood), mild abdominal |
| | distention, occult blood in stool (no fissure) |
| Stage II | Any one or more historical factors |
| (Definite | Above signs and symptoms plus persistant occult or gross |
| NEC) | gastrointestinal bleeding, marked abdominal distention |
| | Abdominal radiographs showing significant intestinal |
| | distention with ileus, small-bowel separation (edema in bowel |
| | wall or peritoneal fluid), unchanging or persistent “rigid” |
| | bowel loops, pneumatosis intestinalls, portal venous gas |
| Stage III | Any one or more historical factors |
| (Advanced | Above signs and symptoms plus deterioration of vital signs, |
| NEC) | evidence of septic shock, or marked gastrointestinal |
| | hemorrhage Abdominal radiographs showing pneumoperi- |
| | toneum in addition to findings listed for Stage II |

PARP

Although NEC, the medical disorder, is well recognized, the specific mechanisms of NEC remain poorly defined. Several mechanisms have been linked to the initiation and/or progression of NEC, including mechanisms that modulate endothelial function in the newborn intestine such as cytokine production, infections, nitric oxide (NO) production, apoptosis, and mitochondrial alterations. NO dysregulation, inflammation, and oxidant stress are known to play an important role in all of these mechanisms.

Endogenous NO production is catalyzed by a family of enzymes known as nitric oxide synthase (NOS). The isoform most commonly implicated in NO dysregulation is NOS 2 (also known as inducible NOS or iNOS). NO is produced in the intestine and under physiologic conditions serves as a mediator of intestinal tone and contractility. In addition, NO acts as an intercellular transduction mediator, intracellular effector of enzymes, and as a toxicant for immune defense. These divergent roles suggest that strict control of intra- and intercellular levels of NO are critical, and loss of NO regulation may have drastic consequences. In some instances, superoxide anion destroys NO, reducing its efficacy as a signal transduction agent, and promoting the formation of peroxynitrite, a highly reactive nitrogen species known to nitrate protein tyrosine residues and cause cellular oxidative damage, including DNA strand breaks. Excess or uncontrolled production of reactive nitrogen species production can shift the actions of available NO from a useful cellular signal to a toxic free radical.

Widespread NOS II (nitric oxide synthase II) induction and protein nitration have been found in human NEC specimens and it is known that reactive nitrogen species also cause DNA damage. Following DNA damage due to oxidation, the enzyme poly(ADP-ribose) polymerase (PARP), a DNA repair enzyme, is activated.

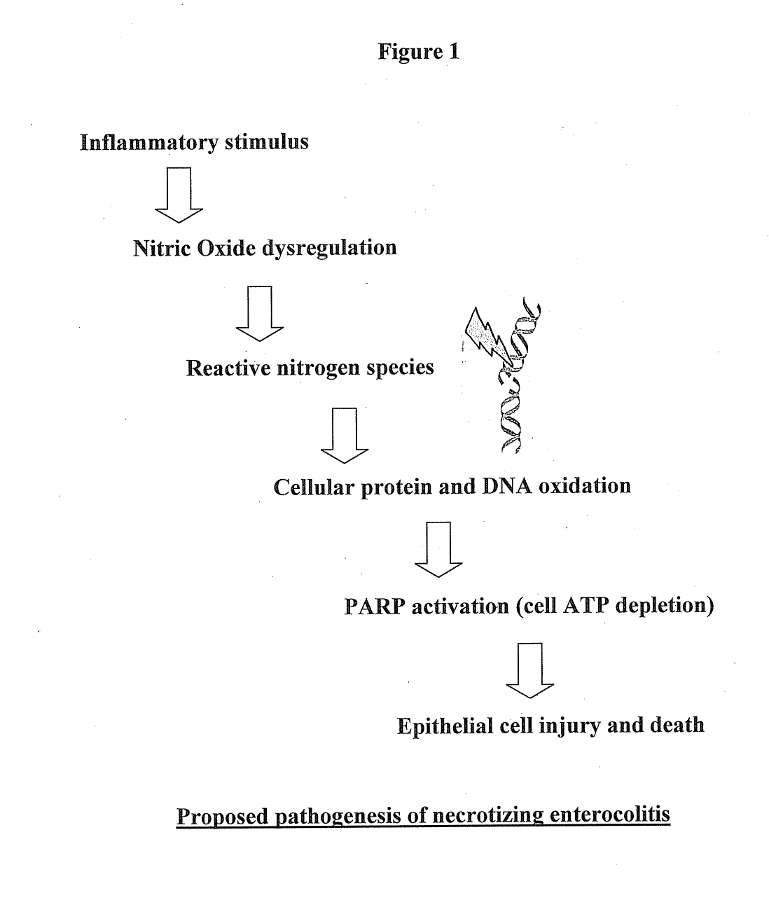

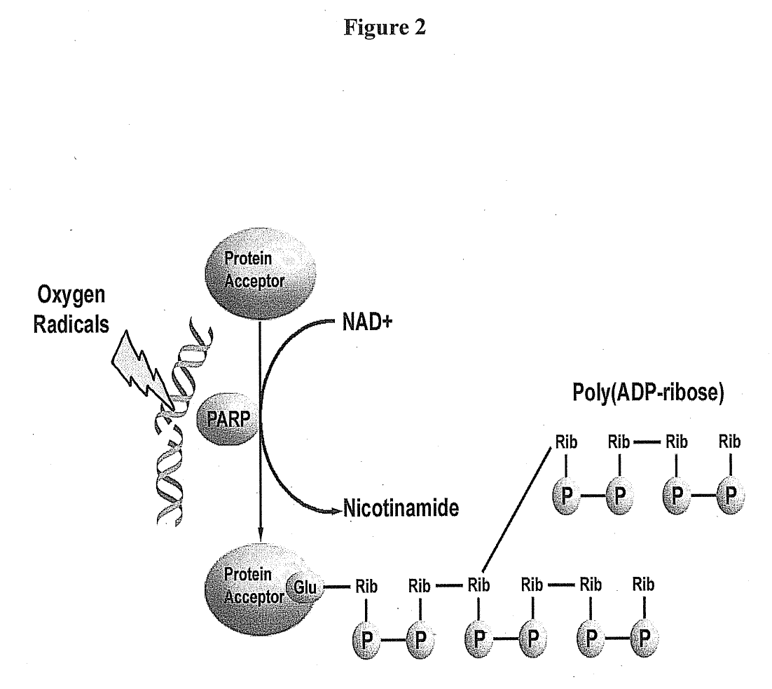

Poly(ADP ribose) polymerase (PARP), which is also referred to as ADPRT (NAD:protein (ADP-ribosyl transferase (polymersing)) and PARS (poly(ADP-ribose) synthetase), is an abundant nuclear enzyme which is activated by DNA strand single breaks to synthesize poly (ADP ribose) from NAD. Under normal conditions, PARP is involved in base excision repair caused by oxidative stress via the activation and recruitment of DNA repair enzymes in the nucleus. Thus, PARP plays a role in cell necrosis and DNA repair. PARP also participates in regulating cytokine expression that mediates inflammation. Under conditions where DNA damage is excessive, PARP is over-activated, resulting in cell-based energetic failure characterized by NAD depletion and leading to ATP consumption, cellular necrosis, tissue injury, and organ damage/failure. (FIGS. 1 and 2)

PARP is an enzyme activated to facilitate DNA repair. It uses nicotinamide adenine dinucleotide (NAD+) as a substrate and attaches ADP-ribose (PAR) units to itself and other acceptor proteins. This poly(ADP-ribosyl)ation allows the acceptor proteins to selectively influence important cellular responses that enhance DNA repair and transcription of inflammatory mediators such as Nf-κB. When the PAR polymer becomes too large, it will result in PARP deactivation. In this case, poly(ADP-ribose) glycohydrolase (PARG) may re-activate these proteins by removing the PAR that is being continually added to a PARP or other acceptor proteins, allowing PARP to remain activated and continue to increase PAR production. However, in the presence of severe cellular oxidative stress and DNA damage, over-activation of PARP may ensue. Without wishing to be bound by theory, it is thought that this may lead to cell death by two possible mechanisms. First high PAR turnover via PARP activation may deplete the cells of NAD+/ATP energy stores, killing the cells by metabolic catastrophe. Second, increased PARP activation can lead to the release apoptotic inducing factor (AIF) from the mitochondria leading to apoptosis. Thus, the activity of PARP in an injured or activated cell serves as a checkpoint or governor between the fate of cellular repair and the fate of cell death (via either apoptosis or necrosis).

Without wishing to be bound by theory, it is thought that there are three potential mechanisms by which PARP may contribute to tissue injury and cell death. First, NAD+ consumption by PARP may deplete the cells of energy metabolites leading to cellular necrosis. Second, PARP activation leads to AIR release from mitochondria that causes AIF-mediated caspase-independent apoptotic cell death. Lastly, PARP appears to be part of the NF-κB transcriptosome and thus contributes to the synthesis of inflammatory mediators. All three mechanisms may be involved in NEC.

Because of the role PARP plays in cell death, it is believed that PARP inhibitors can have therapeutic effects and a substantial number of pharmacologic studies have shown the benefit of various classes of PARP inhibitors in many disease models in adults such as inflammation and oxidation (Lobo S M, et al., J Surg Res. 2005 129:292-7), neurodegeneration and vascular disease, including myocardial infarction (Palfi A, et al., J Mol Cell Cardiol. 2006 41:149-59), septic shock (Genovese T, et al., Crit. Care Med. 2004 32:1365-74), and experimental colitis as a model for inflammatory bowel disease (Mazzon E, et al., Biochem Pharmacol. 2002 64:327-37). Some of these inhibitors have entered human trials for adults (Jagtap P, et al., Nat Rev Drug Discov. 2005 4:421-40).

PARP Inhibitors

PARP inhibitors are compounds which inhibit the in vitro and/or in vivo polymerase activity of poly(ADP-ribose) polymerase (PARP), and compositions containing these compounds. PARP inhibitors limit or inhibit PARP activity either locally or systemically.

Many of the PARP inhibitors are known and, thus, can be synthesized by known methods from starting materials that are known, may be available commercially, or may be prepared by methods used to prepare corresponding compounds in the literature. See, for example, Suto et al., “Dihydroisoquinolincnes: The Design and Synthesis of a New Series of Potent Inhibitors of Poly(;SP-ribose) Polymerase”, Anticancer Drug Des., 6:107-17 (1991), which discloses processes for synthesizing a number of different PARP inhibitors. Further processes for synthesizing compounds useful in the methods of the present invention are described in the following international and U.S. patent applications: PCT/U.S. Ser. No. 98/18184, PCT/U.S. Ser. No. 98/18226, PCT/U.S. Ser. No. 98/18187, PCT/U.S. Ser. No. 98/18195, PCT/U.S. Ser. No. 98/18196, PCT/U.S. Ser. No. 98/18188, PCT/U.S. Ser. No. 98/18189, PCT/U.S. Ser. No. 98/18185, PCT/U.S. Ser. No. 98/18186, and U.S. application Ser. Nos. 08/922,520, 09/079,513, 09/145,179, 09/079,508, 09/145,166, 09/079,507, 09/145,177, 09/145,180, 09/079,509, 09/079,510, 09/145,184, 09/079,511, 09/145,185, 08/922,548, 09/145,181, 09/147,502, 09/219,843, 08/922,575, 09/079,512, 09/145,176, 09/079,514, 09/145,178, 09/224,293, 09/224,294 and 09/387,767, the entire contents of each of which are hereby incorporated by reference.

In addition, many PARP inhibitors have been described in Banasik et al., “Specific Inhibitors of Poly(ADP-Ribose) Synthetase and Mono(ADP-Ribosyl)-Transferase”, J. Biol. Chem., 267:3, 1569-75 (1992), and in Banasik et al., “Inhibitors and Activators of ADP-Ribosylation Reactions”, Molec. Cell. Biochem, 138:185-97 (1994), the entire contents of which are incorporated herein by reference.

Examples of suitable PARP inhibitors include, but are not limited to, nicotinamide, 3-aminobenzamide, picolinamide, 5-methyl nicotinamide, methylxanthines, thymidine, benzamide, 3-methoxybenzamide, 4-aminobenzamide, 2-aminobenzamide, pyrazinamide, theobromine, theophylline, 3-aminophtalhydrazide and 1,5-dihydroxyisoquinoline or a mixture thereof. U.S. Pat. No. 5,756,510 (Griffin et al.), the entire contents of which are incorporated herein by reference, discloses benzamide analogs having PARP inhibitory efficacy and those compounds may be used in the present invention methods and compositions. Other suitable PARP inhibitors are described in Ferraris, Dana V. et al., United States Patent Application 2006/0003987, filed Aug. 30, 2005, the entire contents of which are incorporated herein by reference. Other examples of PARP inhibitors that may be used in the present invention include: chemical derivatives of nicotinamide, or compounds belonging to the known class of 6(5H)phenanthridinone (described in U.S. Pat. Nos. 6,476,048 and 6,531,464 to Szabo et al., the entire contents of which are incorporated herein by reference); or belonging to the class of isoquinoline derivatives, (described in U.S. Pat. No. 7,268,143 to Jagtap et al., the entire contents of which are incorporated herein by reference); or substituted isoindolinone and derivatives (described in U.S. Pat. No. 6,534,651 to Jagtap et al., the entire contents of which are incorporated herein by reference); or substituted tetracycline benzamide (or substituted indeno[1,2-c]isoquinoline derivatives, as described in U.S. Pat. No. 6,828,319 to Jagtap et al., the entire contents of which are incorporated herein by reference.

The PARP inhibitors of the present invention may also be determined by various assays described in the literature. The following publications teach various methods which may be employed to elucidate PARP inhibitors: 1) U.S. Pat. No. 5,756,510 (Griffin et al.); 2) Banasik et al. Specific inhibitors of poly(ADP-ribose) synthetase and mono(ADP-ribose) transferase., J Biol Chem, volume 267, pages 1569-1575 (1992); and 3) Schanraufstatter et al. Oxidant injury of cells. DNA strand-breaks activate polyadenosine diphosphate-ribose polymerase and lead to depletion of nicotinamide adenine dinucleotide., J Clin Invest, volume 77, pages 1312-1320 (1986). In some examples, the therapeutically effective amount is the amount of PARP inhibitor which will achieve at least a 20% reduction in PARP activity as compared to a control. IN some examples, the control is a tissue that has not been treated with a PARP inhibitor

Typically, the PARP inhibitors contemplated by the invention will have an IC50 for inhibiting poly(ADP-ribose) polymerase in vitro that is equal to or less than 100 μM, 75 μM, 50 μM, 20 μM, 10 μM, 1 μM, 0.1 μM, or 0.01 μM.

Thus, some embodiments relate to a method of treating a human neonate who has the clinical signs or symptoms of NEC, or who is at risk of developing NEC, a therapeutically effective amount of a pharmaceutical composition comprising one or more PARP inhibitors.

For this purpose, the PARP inhibitors may be useful in the free base form, in the form of base salts where possible, and in the form of addition salts, as well as in the free acid form. All these forms are within the scope of this invention. In practice, use of the salt form amounts to use of the base form. Pharmaceutically acceptable salts within the scope of this invention are those derived from mineral acids such as hydrochloric acid and sulfuric acid; and organic acids such as ethanesulfonic acid, benzenesulfonic acid, p-toluenesulfonic acid, and the like, giving the hydrochloride, sulfonate, ethanesulfonate, benzenesulfonate, p-toluenesulfonate, and the like respectively, or those derived from bases such as suitable organic and inorganic bases. Examples of pharmaceutically acceptable base addition salts with compounds of the present invention include organic bases which are nontoxic and strong enough to form such salts. These organic bases and the use thereof are readily understood by those skilled in the art. Merely for the purpose of illustration, such organic bases may include mono-, di-, and trialkylamines, such as methylamine, diethylamine and triethylamine; mono-, di-, or trihydroxyalkylamines such as mono-, di-, and triethanolamine; amino acids such as arginine, and lysine; guanidine; N-methylglucosamine; N-methylgiucamine; L-glutamine; N-methylpiperazine; morpholine; ethylenedianane; N-benzylphenethylamine; tris (hydroxymethyl) antinoethane; and the like.

The acid addition salts of the basic compounds may he prepared by dissolving the free base of the compounds of the present invention in aqueous or aqueous alcohol solution or other suitable solvents containing the appropriate acid or base and isolating the salt by evaporating the solution, or by reacting the free base of a compound of the present invention with an acid as well as reacting a compound of the present invention having an acid group thereon with a base such that the reactions are in an organic solvent, in which case the salt separates directly or can be obtained by concentration of the solution.

The PARP inhibitors typically contain one or more asymmetric carbon atoms. Therefore, the invention includes the individual stereoisomers and mixtures thereof as well as the racemic compounds. The individual isomers may be prepared or isolated by methods known in the art.

Also included as useful compounds in the present methods are the pharmaceutically acceptable salts, hydrates, esters, and solvates of the PARP inhibitors and derivatives described herein.

“Pharmaceutically acceptable salt”, “hydrate”, “ester” or “solvate” refers to a salt, hydrate, ester, or solvate of the inventive compounds which possesses the desired pharmacological activity and which is neither biologically nor otherwise undesirable. Organic acids can be used to produce salts, hydrates, esters, or solvates such as acetate, adipate, alginate, aspartate, benzoate, benzenesulfonate, p-toluenesulfonate, bisulfate, sulfamate, sulfate, naphthylate, butyrate, citrate, camphorate, camphorsulfonate, cyclopentane-propionate, digluconate, dodecylsulfate, ethanesulfonate, fumarate, glucoheptanoate, glycerophosphate, hemisulfate heptanoate, hexanoate, 2-hydroxyethanesulfonate, lactate, maleate, methanesulfonate, 2-naphthalenesulfonate, nicotinate, oxalate, tosylate and undecanoate. Inorganic acids can be used to produce salts, hydrates, esters, or solvates such as hydrochloride, hydrobromide, hydroiodide, and thiocyanate.

Examples of suitable base salts, hydrates, esters, or solvates include hydroxides, carbonates, and bicarbonates of ammonia, alkali metal salts such as sodium, lithium and potassium salts, alkaline earth metal salts such as calcium and magnesium salts, aluminum salts, and zinc salts.

Salts, hydrates, esters, or solvates may also be formed with organic bases. Organic bases suitable for the formation of pharmaceutically acceptable base addition salts, hydrates, esters, or solvates of the compounds of the present invention include those that are non-toxic and strong enough to form such salts, hydrates, esters, or solvates. For purposes of illustration, the class of such organic bases may include mono-, di-, and trialkylamines, such as methylamine, dimethylamine, triethylamine and dicyclohexylamine; mono-, di- or trihydroxyalkylamines, such as mono-, di-, and triethanolamine; amino acids, such as arginine and lysine; guanidine; N-methyl-glucosamine; N-methyl-glucamine; L-glutamine; N-methylpiperazine; morpholine; ethylenediamine; N-benzyl-phenethylamine; (trihydroxy-methyl)aminoethane; and the like. See, for example, “Pharmaceutical Salts,” J. Pharm. Sci., 66:1, 1-19 (1977). Accordingly, basic nitrogen-containing groups can be quaternized with agents including: lower alkyl halides such as methyl, ethyl, propyl, and butyl chlorides, bromides and iodides; dialkyl sulfates such as dimethyl, diethyl, dibutyl and diaryl sulfates; long chain halides such as decyl, lauryl, myristyl and stearyl chlorides, bromides and iodides; and aralkyl halides such as benzyl and phenethyl bromides.

The acid addition salts, hydrates, esters, or solvates of the basic compounds may be prepared either by dissolving the free base of a PARP inhibitor of the present invention in an aqueous or an aqueous alcohol solution or other suitable solvent containing the appropriate acid or base, and isolating the salt by evaporating the solution. Alternatively, the free base of the PARP inhibitor of the present invention can be reacted with an acid, as well as reacting the PARP inhibitor having an acid group thereon with a base, such that the reactions are in an organic solvent, in which case the salt separates directly or can be obtained by concentrating the solution.

For the purpose of treating or preventing NEC, the composition may be administered orally, parenterally, by inhalation spray, adsorption, absorption, or rectally. The term parenteral as used herein includes subcutaneous, intravenous, intramuscular, and intraperitoneal, injection or infusion techniques.

When administered parenterally, the composition will normally be in a unit dosage, sterile injectable form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic: parenterally-acceptable diluents or solvents, for example, as solutions in 1,3-butanediol. Among the acceptable vehicles and solvents that may be employed are water, saline, Ringer's solution, dextrose solution isotonic sodium chloride solution, and Hanks' solution. In addition, sterile, bland fixed oils are conventionally employed as solvents or suspending mediums. These oil solutions or suspensions may also contain long chain alcohol diluents or dispersants.

The PARP inhibitors contemplated herein may be used together with one or more pharmaceutically acceptable carriers or excipients, and optionally other therapeutic ingredient(s). In some examples, the carriers or excipients are added to enhance the solubility or stability of the product used to treat NEC.

The composition of the invention may be administered orally or via an enteral feeding tube as a liquid, aqueous suspension or solution, containing a single or divided dose of the inhibitor.

The compounds of this invention may also be administered rectally in the form of suppositories. These compositions can be prepared by mixing the drug with a suitable non-irritating excipient which is solid at room temperature, but liquid at rectal temperature, and, therefore, will melt in the rectum to release the drug. Such materials include cocoa butter, beeswax, and polyethylene glycols.

One PARP inhibitor is nicotinamide. Nicotinamide is one of the two principal forms of the B-complex vitamin niacin and is also known as niacinamide, 3-pyridinecarboxamide, pyridine-3-carboxamide, nicotinic acid amide, vitamin B3 and vitamin PP. Nicotinamide has been classed by FDA as a food additive under the moniker Generally Recognized As Safe or GRAS and is the principal form of niacin used in nutritional supplements and in food fortification. Its molecular formula is C₆H₆N₂O and its molecular weight is 122.13 daltons and the structural formula is:

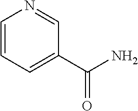

Nicotinamide has a wide safety profile in humans. The 50% inhibition concentration of nicotinamide for PARP is nearly 0.1 mM.

The plasma concentration of PARP inhibitors depends on a number of factors, including but not limited to, the route of administration, the metabolic state of the neonate, and the maturity (i.e. whether preterm, term, etc.) of the neonate. Thus, an oral dose of a PARP inhibitor may achieve different plasma concentration in different subjects.

Nicotinamide can be administered in doses which will attain a plasma concentration of 0.1-10 mM. The plasma nicotinamide concentration in some examples include: 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, or any range in between.

The route of administration can be oral, enteral (via a feeding tube), rectal (via a suppository), intravenous, intra-arterial (e.g. though the umbilical cord), intramuscular, intra-peritoneal, subcutaneous, or other parenteral routes.

Nicotinamide can be used in doses ranging from 5 mg/kg to 500 mg/kg. Examples of nicotinamide dosage includes 5-10 mg/Kg, 10-20 mg/Kg, 20-50 mg/Kg, 50-75 mg/Kg, 75-100 mg/kg, 100-200 mg/Kg, 100-300 mg/Kg, 100-400 mg/Kg, or 100-500 mg/Kg. In some examples, the dose is 25-45 mg/Kg, 30-35 mg/Kg, or 30 mg/Kg. The doses can be administered as slow release, once daily, or as multiple daily doses. Doses of 500 mg/kg or more have been used in animal preparations with little or no reported toxicities.

The administered dose of nicotinamide can be expressed in units other than as mg/Kg. For example, doses can be expressed as mg/m². One of ordinary skill in the art would readily know how to convert doses from mg/kg to mg/m² given either the height or weight of a subject or both (see e.g., http:///www.fda.gov/cder/cancer/aiiimalframe.htm). For example, a dose of 50 mg/Kg in a 65 kg human is approximately equal to 1.9 g/m². The same dose of 50 mg/Kg for a 2 Kg nenoate is approximately 600 mg/m²

TABLE III
Comparison of niacin (nicotinamide) doses in various preparations

| | | | | Present |
|---|---|---|---|---|
| | | | Present | invention: |
| | | | invention: | High-dose |
| | | | Nicotinamide | nicotinamide |
| Recommended | | | for treatment | infant food |
| daily intake | In breast | | or prevention | (e.g. formula) |
| (RDI) (USA) | milk | In formula | of NEC | 5-500 × RDI |
| 6 mg/day | 1.8-3.9 | 3.4-9.0 | 5-500 mg/Kg | 30-3000 |
| | mg/L | mg/L | | mg/day, |
| | (av = 0.33 | (0.75-1.25 | | Or |
| | mg/100 | mg/100 | | 17-4500 mg/L |
| | kcal) | kcal) | | |

In some instances, effective therapy for NEC involves the administration of PARP inhibitors, such as nicotinamide, very early in the course of the disease. In other instances, administration of the PARP inhibitor would begin before or at the time of birth of an infant at risk for developing NEC as a prophylactic measure to prevent the onset of NEC.

Infant Food and Treatment Compositions

In another embodiment, the present invention relates to an infant food or treatment composition that comprise a high dose of PARP inhibitor, i.e. in an amount that is 5 to 500 times greater than the daily recommended intake (RDI) dosage for the PARP inhibitor. Ranges for the PARP inhibitor substances include: 50-250, 50-100, 5-75, 5-50, 5-25, 5-10, 10-25, 25-45, or 30-35 times greater than the RDI. Such a composition would be suitable for use in neonates that are at risk of developing NEC, as a preventative regime; or in neonates that already have NEC, as a treatment regime.

### Infant Formula

In one embodiment, the infant food composition is a high dose PARP inhibitor infant formula. PARP inhibitors have been described in detail above. In one example, the PARP inhibitor is nicotinamide.

In one example, the present formula is a high dose nicotinamide infant formula for pre-term or term babies. Such an infant formula includes nicotinamide in the following stated amounts: 17-45000 mg/L, 17-3000 mg/L, 20-1000 mg/L, 20-500 mg/L, 20-300 mg/L, 20-150 mg/L, 30-150 mg/L, 30-100 mg/L, 100-500 mg/L, or 150-450 mg/L.

The present infant formula includes a nutritionally balanced formulation which will generally comprise a source of protein, carbohydrates, edible fats, and water. The infant formula.

The present formula may also contain one or more vitamins and minerals essential in the diet of the preterm or term infant. These vitamins and minerals should be present in nutritionally significant amounts. Examples of vitamins and minerals which may be added to the present infant formula compositions include vitamin A, vitamin B complex, vitamin C, vitamin D, vitamin E, vitamin K, calcium, magnesium, sodium, potassium, phosphorous, copper, zinc, chloride, iodine, selenium, iron, niacin, folic acid, pantothenic acid, biotin, choline, inositol and manganese.

The infant formula compositions of the present invention may further comprise macronutrients beneficial to the preterm infant such as carotenoids, taurine, coconut oil, sunflower oil, soy oil, native or synthetic fats, palmitic acid, medium chain triglycerides, lactose, maltodextrin, glucose, bovine oligosaccharides or fractions thereof, skim milk, milk whey, soy protein, rice protein, protein hydrolysates and lactoferrin.

In some examples, the present infant formulas are particularly suited for feeding the preterm or low birth weight infant. The premature infant is notably different from the term infant. The initial immaturity and subsequent rapid growth rate of the premature infant necessitates a higher nutrient intake than the term infant. The composition of preterm infant formula is based primarily on the composition of human breast milk and on intra-uterine accretion. Preterm infant formulas have thus been modified to contain appropriately increased amounts of nutrients, such as protein and fat, as well as minerals, such as calcium, phosphorus, copper and zinc. Such preterm infant formulas may contain, in addition to the nutrients states above, nucleotides at levels found in breast milk of mothers of preterm infants, which are quite different than the levels of nucleotides found in the breast milk of mothers of term infants. Examples of such preterm infant formulas are well in the art. See, for example, US PAT APP: 2005/0058690.

Infant formulas for term babies are also well known in the art and contain ingredients that closely resemble those in human breast milk. Examples of these formulas are enumerated in U.S. Pat. No. 4,994,442, the contents of which are incorporated herein by reference.

The present infant formulas may be in the form of a liquid, either as a ready to feed liquid or as a concentrated liquid requiring dilution with water prior to feeding, or in a powder form requiring reconstitution with water prior to use. The present infant formulas may be either milk-based or not milk-based. In general, infant formulas, according to the present invention have a composition adequate for meeting the requirements of low birth weight infants, at term infants, infants with lactose intolerance, infants with cow's protein intolerance and/or malabsorption syndrome.

The present infant formulas may be prepared according to methods well known in the art. In one example, the present infant formula may be prepared by blending appropriate quantities of whey protein concentrate with skimmed milk, lactose, vegetable oils and fat soluble vitamins in deionized water. Preferably, these materials are blended together in quantities sufficient to provide a final concentration of approximately 240 grams/liter. Mineral salts may then be added to the mixture prior to a high temperature/short time pasteurization step. Appropriate mineral salts include calcium chloride, calcium phosphate, sodium citrate, potassium hydroxide, potassium bicarbonate, ferrous sulfate, zinc sulfate, sodium chloride, copper sulfate, potassium iodide, sodium selenite, etc. The mixture is then homogenized and cooled. Heat-labile vitamins and micronutrients may then be added to the mixture. The mixture is then standardized with deionized water to a final total solids concentration of about 150 to about 160 and preferably about 155 grams per litre, which is equivalent to about 820 kcal per liter. The formula may be sterilized using a conventional ultrahigh temperature process, and then aseptically filled into appropriate packaging. Alternatively, the formula may be filled into glass jars and then retort sterilized.

It would be recognized by one skilled in the art that other known methods of manufacture and sterilization can be used for the preparation of the present infant formula. The present infant formula may also be produced as a concentrated liquid product requiring dilution with an equal volume of water prior to feeding to an infant. Furthermore such an infant formula may be dehydrated, such as in a spray dryer, to create a stable infant formula powder that offers advantages of stability and economy of transport, said powder requiring reconstitution with water prior to feeding to an infant.

Food Supplements

Also contemplated herein is a high dose PARP inhibitor food supplement formulated to be used for: oral consumption or enteral feeding. PARP inhibitors have been described in detail above. In one example, the PARP inhibitor is nicotinamide.

In one example, the food supplement will include a high dose nicotinamide formulation suitable for consumption by a pre-term neonate, or a tem-neonate at risk of developing NEC. Such a food supplement may also include other carriers, excipients, etc. as necessary. In some examples, such a neonatal food supplement includes nicotinamide in the following stated amounts: 17-45000 mg/L, 17-3000 mg/L, 20-1000 mg/L, 20-500 mg/L, 20-300 mg/L, 20-150 mg/L, 30-150 mg/L, 30-100 mg/L, 100-500 mg/L, or 150-450 mg/L.

Treatment Compositions

Also contemplated herein is a high dose PARP inhibitor composition prepared for administration by a route other than oral.

In one example, the composition is an enteral, intra-arterial (IA), intravenous (IV), intramuscular (IM), intraperitoneal (IP), subcutaneous (SC), or rectal (suppository) preparation that includes a PARP inhibitor and a pharmaceutically acceptable carrier or excipient.

The PARP inhibitor is present in an amount that will achieve plasma concentrations that are shown to inhibit PARP activity by at least 20%. In some examples, the PARP inhibitor will have an IC50 for inhibiting poly(ADP-ribose) polymerase in vitro that is equal to or less than 100 μM, 75 μM, 50 μM, 20 μM, 10 μM, 1 μM, 0.1 μM, or 0.01 μM.

In one example, the PARP inhibitor in the above mentioned preparations is nicotinamide. In some examples, the amount of nicotinamide in the compositions is such that when administered, it will attain a plasma concentration of 0.1-10 mM. The plasma nicotinamide concentration in some examples include: 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, or any range in between

The present invention will be better understood by reference to the following examples which are offered by way of illustration not limitation.

### Example 1

### Evidence of Reactive Nitrogen Species Formation in the Rat NEC Model

Materials and Methods: Rodent Model of NEC for Preclinical Research

We have established valuable preclinical animal models that mimic intestinal injury in NEC. (Caplan M S, Hedlund E, Adler L, et al. Role of asphyxia and feeding in a neonatal rat model of necrotizing enterocolitis. Pediatr Pathol. November-December 1994; 14(6):1017-1028.) The model involves exposing neonatal rat pups to hypoxia, hypothermia, and formula feeding, leading to physiological stress and intestinal injury that pathologically is very similar to NEC in infants. Pups are delivered by cesarean section at E20 from timed pregnant dams. Pups are fed every 3 h with one skipped feed at 4 am (7 times/day) by oral gavage with 100 ul of a hypertonic formula (Similac and Esbilac). At 8 h post-delivery, pups are treated with 2 mg/kg LPS orally with feeding. Beginning on DOL 1, pups are fed every 3 h and subjected to hypoxic (2 min in 100% nitrogen) and hypothermal (10 min at 4° C.) stress twice daily immediately after feeds. Pups are sacrificed at DOL 4, or when discomfort warrants. Upon sacrifice, the distal ileum is collected in two sections. One section is immediately fixed in 10% neutral buffered formalin, and the other is snap-frozen in liquid nitrogen. Tissues are fixed for 48 h before histological processing and embedding in paraffin.

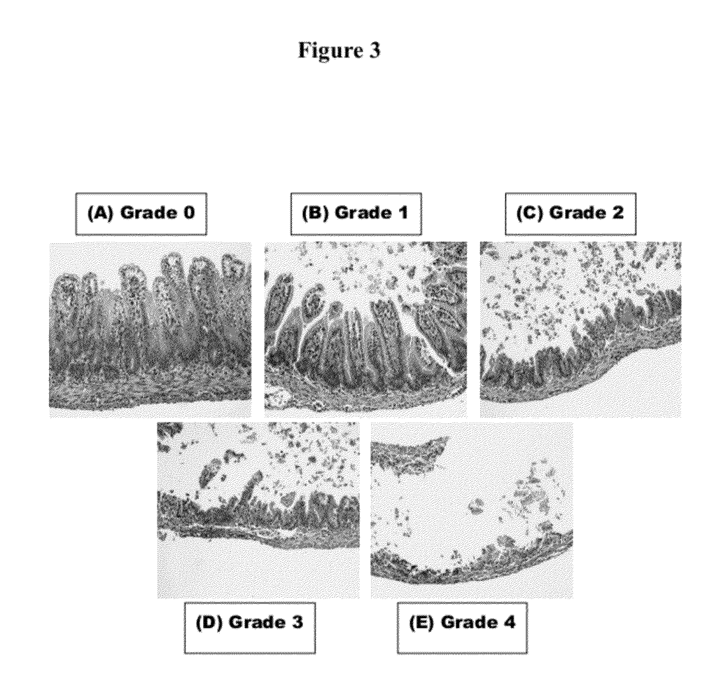

Shown in FIG. 3 are representative photomicrographs of newborn rat intestine (ileum) from control pups (A) and from newborn pups exposed to formula-hypoxia-hypothermia stress for 4 days (B-E). Each photomicrograph represents a different severity grading score of intestinal injury in this model as follows: (A)=Grade 0 or normal, (B)=Grade 1 or minimal intestinal damage, (C)=Grade 2 or minimal/modest intestinal damage, (D)=Grade 3 or moderate intestinal injury, (E) Grade 4 or severe intestinal injury. This system is based on the previously published intestinal injury grading system for the newborn rat intestinal model of NEC induced by formula feeding-hypoxia-hypothermia. These photomicrographs demonstrate our ability to perform the newborn rat model of NEC and to score the injury that has incurred.

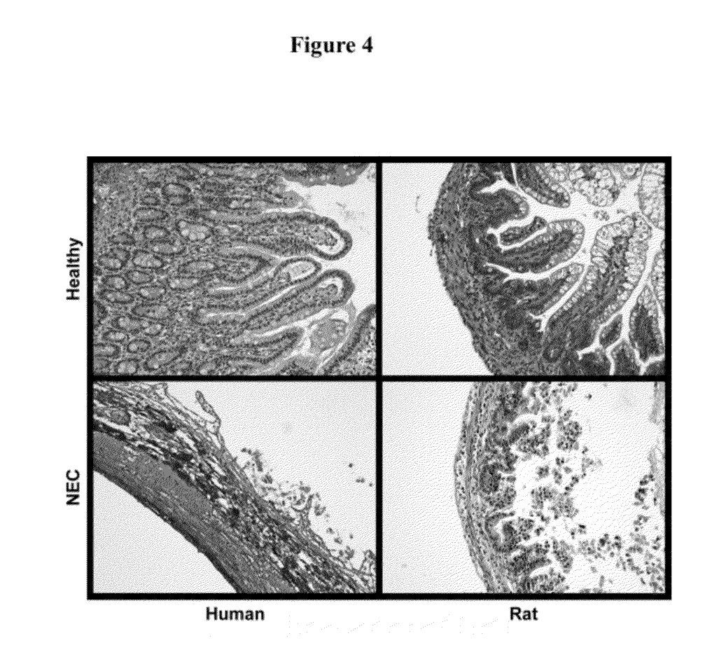

FIG. 4 shows representative images of normal and NEC tissues from the rat model and human samples, showing the histologic similarities between the diseases. The general morphological aspects are identical; therefore the methods that we have developed for detailed region-specific imaging will be valuable in both tissue settings.

Results

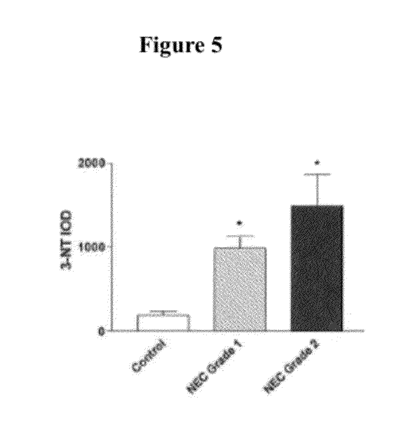

Using immunohistochemistry, we have studied the prevalence of protein 3-nitrotyrosine (3-NT, a stable biomarker of peroxynitrite and other reactive nitrogen species) in ileal tissues from rats with Grade 1 and Grade 2 NEC. In these experiments, we focused on these Grades since they represent the early transition to severe tissue injury and cell death, thus they are relevant for design of pharmacological interventions to prevent progression and/or enhance recovery. Digital image analysis showed evidence of increased 3-NT in the proximal villus region, demonstrating an increased prevalence of oxidative cellular injury. (FIG. 5) This region of enterocytes is critical for restitution from the initial stages of NEC and is therefore considered a specific region to achieve pharmacological efficacy of protection/survival [*, p<0.05 versus control].

### Example 2

### First Evidence of PARP Activation in the Rat NEC Model

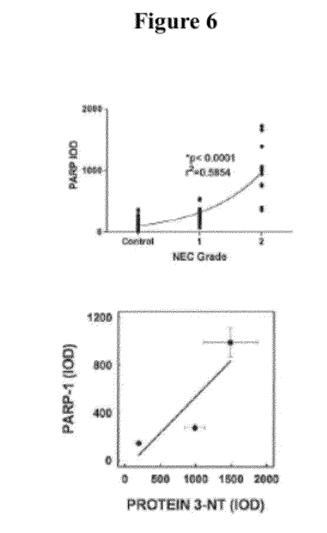

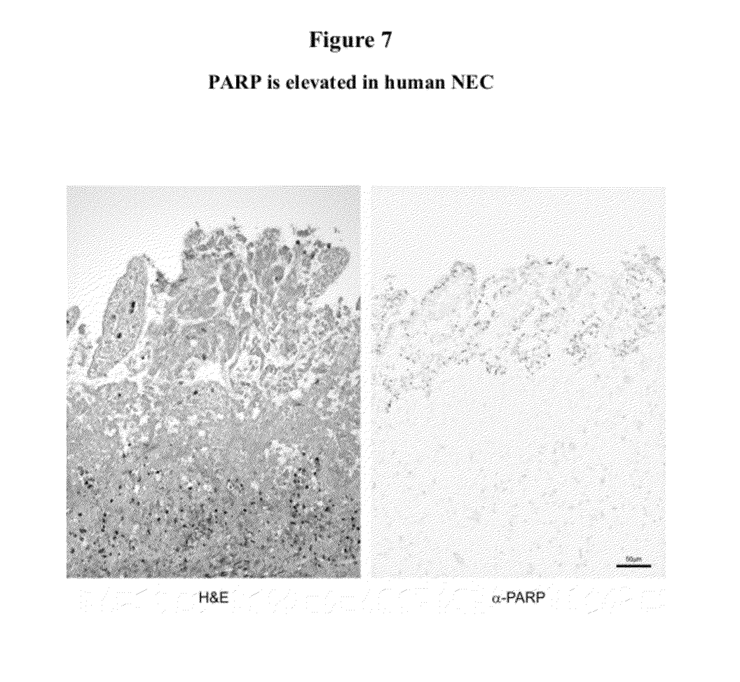

Previous studies by others have shown that an important consequence of increased reactive nitrogen species is the activation of PARP and ensuing cellular necrosis and/or apoptosis. In the context of NEC, we formed the novel hypothesis that PARP is increased in proximal villus regions during early stages of NEC. We therefore used immunohistochemical approaches to investigate the prevalence of PARP-1 in rat ileal tissues with Grade 1 and Grade 2 NEC. Using these approaches, we have observed a striking increase in the levels of PARP-1 in the same regions of tissue that we observed protein nitration. (FIG. 6) Furthermore, we observed a significant positive association of PARP-1 prevalence in the regions of highest protein nitration among the NEC tissues investigated. These data strongly support the concept that reactive nitrogen species leads to PARP activation and subsequent cell death. Our data supports the hypothesis that not only is PARP elevated in the proximal villus of newborn rats exposed to the model of NEC, but it appears to be elevated exponentially from Grades 0 to 2, and may be at least in part responsible for the severe intestinal injury and bowel necrosis seen in Grades 3 and 4 and in the later stages of the human disease. (FIG. 7, showing that PARP is elevated in human NEC)

### Example 3

### Nicotinamide is Protective Against Intestinal Injury in a Newborn Rat Model of NEC

Rat pups were given one oral dose of 500 mg/kg of nicotinamide (n=8), 50 mg/kg of nicotinamide (n=8), or vehicle (n=7) each day and placed in the newborn rat NEC protocol. Nicotinamide administration was strikingly protective against the development of any NEC and protected against more severe grades of injury (Table II). Table II shows that Pups treated with nicotinamide have significantly less evidence of any intestinal injury than controls when exposed to the rat model of NEC. (*=p<0.05; One-Way ANOVA). Pups treated with nicotinamide tended to have less severe intestinal injury.

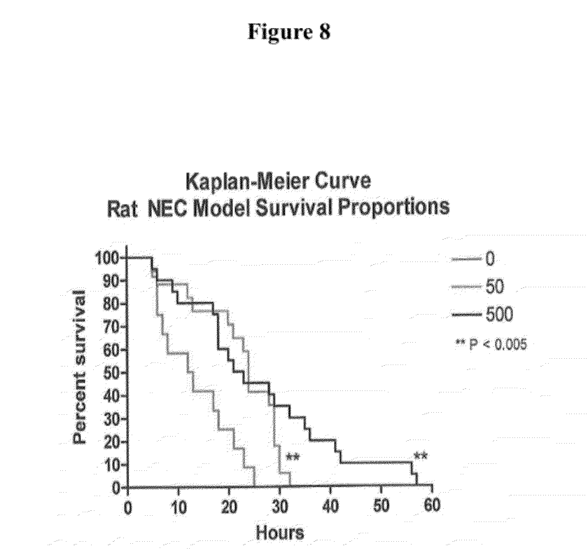

Nicotinamide administration also improved overall survival of pups exposed to the rat NEC model (FIG. 8). FIG. 8 shows that pups treated with nicotinamide have significantly greater survival than control pups when exposed to the rate model of NEC. The pups that received one oral dose daily of nicotinamide at 500 mg/kg had no evidence of NEC Grade 2 or greater.

TABLE II

| | | Nicotinamide | Nicotinamide |
|---|---|---|---|
| | Untreated | 50 mg/kg | 500 mg/kg |
| Percent of Pups with | 57% | 12.5%* | 0%* |
| NEC (any grade) | | | |
| Percent of Pups with NEC | 42% | 12.5% | 0% |
| Grade >2 | | | |

*= p < 0.05, One-Way Anova

### Example 4

### Investigation of Fetal Human Epithelial Cell Response to Injury

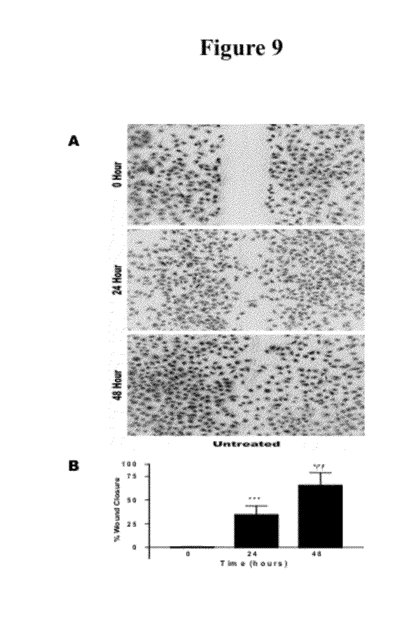

We have established cell culture conditions for neonatal human intestinal epithelial cells (IEC), which we have developed as a platform for investigating cellular responses and identifying potential protection strategies. Human fetal intestinal epithelial cells (FHS741NT), passage seven or less, were seeded (40,000 cells/ml) in poly-1-lysine coated 8-chamber slides (Labtek II; Nunc, Rochester, N.Y.) in media (DMEM containing 10% FBS, L-glutamine (2 mM), EGF (30 ng/ml), and insulin (10 μg/ml)) and allowed to reach confluence. A wound was created in the cell monolayer using a specific cell scraper device attached to a microscope. The microscope stage controls allow the creation of a reproducible straight wound in each slide chamber (width 500 μm). After wounding, media was replaced with media (DMEM with 10% FBS, L-glutamine (2 mM)) containing the appropriate treatment compound. Cells were fixed in 5% neutral buffered formalin after 0, 24, 48, 72 and 96 hrs and stained with crystal violet (FIG. 9A, B).

Digital images of wounds were captured using a 10× objective on an inverted microscope (Olympus, Melville, N.Y.) with an attached digital camera (Diagnostic Instruments, Sterling Heights, Mich.). Image analysis was performed using Image Pro Plus software (Media Cybernetics, Silver Springs, Md.) and a custom-written macro. A rectangular area of interest (AOI) was created and saved based on the 0 hour wound to delineate the initial wound area. Threshold analysis was used to discriminate areas occupied by cells from the background within the image. For each subsequent image, the AOI was placed over the position of the original wound and the area occupied by cells was measured. Data were normalized to time-point matched unwounded controls in order to control for differences in confluence within and between wells. Results were expressed as mean % closure where % closure=area of wound occupied by cells/area of original wound. This measurement was found to be highly reproducible and provides the ability to study cellular response to a ‘stimulus’. Using this approach we can evaluate the effects of inflammatory pathway activation (see below) and protection strategies to enhance restitution.

Inflammatory Activation Delays Restitution of IEC In Vitro.

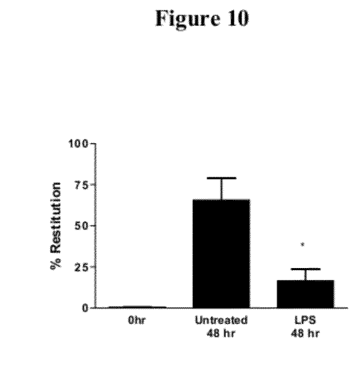

We tested the hypothesis that lipopolysaccharide (LPS), a wall component of gram negative bacteria and potent inflammatory stimulus, exposure (100 μg/ml) prior to cellular injury (scraping) affects epithelial cell restitution. We found that LPS caused striking delays in closure of the scraped wound (FIG. 10), demonstrating that stimulation of inflammatory pathways impairs the ability of these neonatal human cells to repair regions of injury. This concept is consistent with the current view of critical components involved in NEC pathogenesis.

Poly(ADP-Ribose) Formation is Seen when Injured Fetal Intestinal Epithelial Cells are Exposed to LPS.

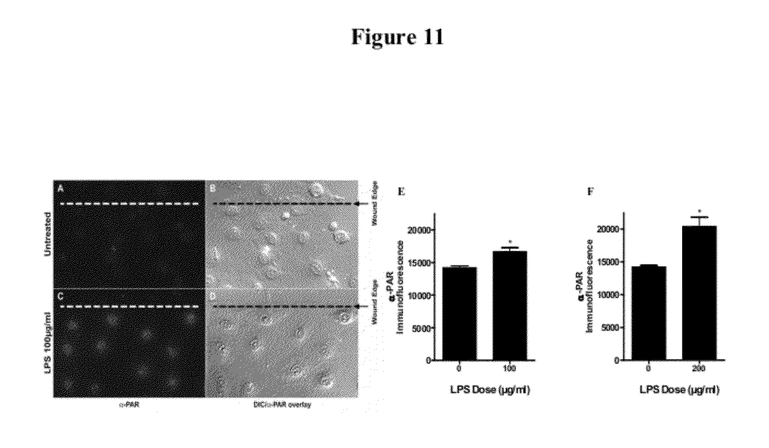

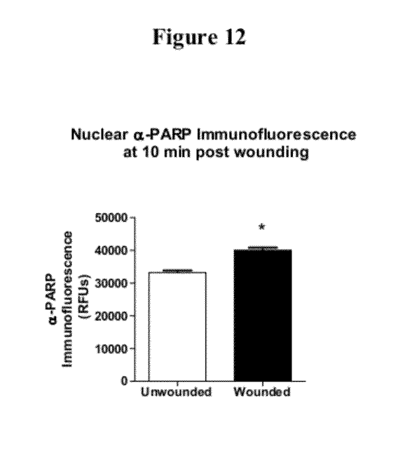

We tested the hypothesis that LPS exposure prior to cellular injury was associated with marked PAR formation signifying increased PARP activation. After the cells were treated as described before, immunocytochemisrty was performed with antibodies against PARP and PAR (anti-PARP and anti-PAR; Trevigen, Gaithersburg, Md.; Dilution: 1:1000) and fluorescent micrographs obtained. Panels A and C in FIG. 11 demonstrate the flouresence of PAR positive IEC after wounding in control and treated (100 ug/ml LPS) cells. Panels B and D in FIG. 11 are differential interference contrast (DIC) images of same fields of view shown in A and C overlaid with the fluorescence staining to allow for visualization of the wound edge along with PAR fluorescence. It was observed the level of PAR is particularly higher in the LPS-treated cells (Panel C), and that a paucity of cells migrated past the wound edge in the PAR positive LPS-treated cells (Panel D) compared with controls (Panel B). Consistent with these findings is the observation that LPS treated cells had markedly decreased wound healing, as shown in FIG. 10. FIG. 11 panels E and F represent quantification of the fluorescence of PAR for wounded IECs treated with 100 ug/ml LPS and 200 ug/ml LPS respectively. Treatment with LPS resulted in increased PAR formation. FIG. 12 shows that PARP-1 is elevated prior to PAR formation in our in vitro model.

Relevance of Our In Vitro Model to Newborn Intestinal Injury In Vivo.

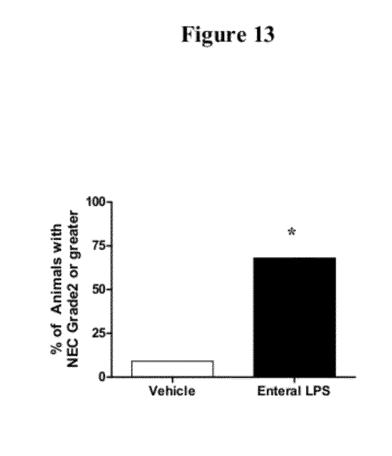

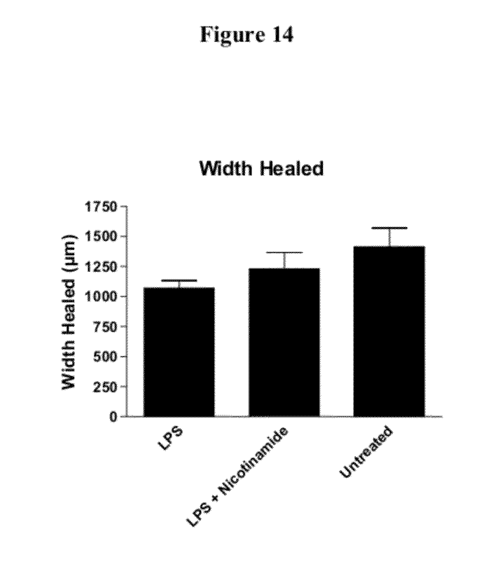

The above studies show that LPS can alter function in fetal IECs. Similar effects were observed in the rat model of newborn NEC, described herein. Addition of enteral LPS (2 mg/kg, p.o.) to the newborn rat NEC model was observed to markedly increase the severity of intestinal injury, as shown in FIG. 13. Treatment of fetal intestinal epithelial cells with 50 mg/ml of nicotinamide after wounding and LPS administration improved wound healing in the in vitro model of immature intestinal wound restitution (FIG. 14).

1. A method for treating or preventing necrotizing enterocolitis (NEC) in a human neonate in need thereof, comprising administering to the neonate a pharmaceutically effective amount of a composition comprising a poly(ADP-ribose) synthetase/polymerase (PARP) inhibitor.

2. The method of claim 1, wherein the neonate in need thereof has NEC and the PARP inhibitor is administered in an amount sufficient to prevent or slow the progression of NEC from an earlier stage to a later stage of the disease.

3. The method of claim 1, wherein the neonate in need thereof is at risk of developing NEC and the PARP inhibitor is administered in an amount sufficient to prevent the clinical onset of NEC in a neonate.

4. The method of claim 1, wherein the neonate in need thereof is preterm.

5. The method of claim 1, wherein the PARP inhibitor is nicotinamide, 3-aminobenzamide, picolinamide, 5-methyl nicotinamide, methylxanthines, thymidine, benzamide, 3-methoxybenzamide, 4-aminobenzamide, 2-aminobenzamide, pyrazinamide, theobromine, theophylline, 3-aminophtalhydrazide, 1,5-dihydroxyisoquinoline or a mixture thereof.

6. The method of claim 1, wherein the administration is via an oral, enteral, rectal, intravenous, intra-arterial, intramuscular, intraperitoneal or subcutaneous route.

7. The method of claim 1, wherein the composition further comprises a pharmaceutically acceptable carrier or excipient.

8. A method for treating a human neonate having necrotizing enterocolitis (NEC) or being at risk of developing NEC, comprising administering to the neonate a pharmaceutically effective amount of a composition comprising nicotinamide.

9. The method of claim 8, wherein the neonate has NEC and the nicotinamide is administered in an amount sufficient to prevent or slow the progression of NEC from an earlier stage to a later stage of the disease.

10. The method of claim 9, wherein the neonate has Grade 1 or Grade 2 NEC.

11. The method of claim 8, wherein the neonate is at risk of developing NEC and the PARP inhibitor is administered in an amount sufficient to prevent the clinical onset of NEC in a neonate.

12. The method of claim 8, wherein the neonate is preterm.

13. The method of claim 8, wherein the therapeutically effective amount of nicotinamide is from 5-500 mg/Kg.

14. The method of claim 8, wherein the administration is via an oral, enteral, rectal, intravenous, intra-arterial, intramuscular, intraperitoneal or subcutaneous route.

15. The method of claim 8, wherein the composition further comprises a pharmaceutically acceptable carrier or excipient.

16. An infant food or treatment composition comprising a PARP inhibitor in an amount that is 5 to 500 times greater than a daily recommended intake dosage for the PARP inhibitor.

17. The composition of claim 16, wherein the composition is a synthetic infant formula for preterm neonates.

18. The composition of claim 16, wherein the composition is a synthetic infant formula for preterm neonates and wherein the PARP inhibitor is nicotinamide in an amount that is 17-4500 mg/L of said formula.

19. The composition of claim 16, wherein the composition is a dietary food supplement for neonates at risk of developing NEC.

20. The composition of claim 16, wherein the composition is a dietary food supplement for neonates at risk of developing NEC and wherein the PARP inhibitor is nicotinamide in an amount that is 17-4500 mg/L.

21. The composition of claim 16, wherein the composition is a pharmaceutical preparation for administration via a non-oral route.

22. The composition of claim 16, wherein the composition is a pharmaceutical preparation for administration via a non-oral route, and wherein the PARD inhibitor is nicotinamide.
